SEQUENCE LISTING (6 sequences)

SEQ ID NO: 1 - DNA, 289 bp, Artificial Sequence
  Synthetic
atttggcgagcgttggttggtggatcaagcccacgcgtaggcaatcctcgagcagatccgccaggcgtgtatatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacgcaaacacaaatacacacactaa

SEQ ID NO: 2 - DNA, 401 bp, Artificial Sequence
  Synthetic
atagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttt

SEQ ID NO: 3 - DNA, 655 bp, Artificial Sequence
  Synthetic
agtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggat

SEQ ID NO: 4 - DNA, 1468 bp, Artificial Sequence
  Synthetic
gccgggatcgaagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactccaagctggccgc

SEQ ID NO: 5 - DNA, 252 bp, Artificial Sequence
  Synthetic
tcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggcc

SEQ ID NO: 6 - DNA, 1267 bp, Artificial Sequence
  Synthetic
gagctcctttcatttctgataaaagtaagattactccatttatcttttcaccaacatattcatagttgaaagttatccttctaagtacgtatacaatattaattaaacgtaaaaacaaaactgactgtaaaaatgtgtaaaaaaaaaatatcaaattcatagcagtttcaaggaatgaaaactattatgatctggtcacgtgtatataaattattaattttaaacccatataatttattatttttttattctaaagtttaaagtaattttagtagtattttatattttgaataaatatactttaaatttttatttttatattttattacttttaaaaataatgtttttatttaaaacaaaattataagttaaaaagttgttccgaaagtaaaatatattttatagtttttacaaaaataaattatttttaacgtattttttttaattatatttttgtatgtgattatatccacaggtattatgctgaatttagctgtttcagtttaccagtgtgatagtatgattttttttgcctctcaaaagctatttttttagaagcttcgtcttagaaataggtggtgtataaattgcggttgacttttaactatatatcattttcgatttatttattacatagagaggtgcttttaattttttaatttttattttcaataattttaaaagtgggtacttttaaattggaacaaagtgaaaaatatctgttatacgtgcaactgaattttactgaccttaaaggactatctcaatcctggttcagaaatccttgaaatgattgatatgttggtggattttctctgattttcaaacaagaggtattttatttcatatttattatattttttacatttattttatatttttttattgtttggaagggaaagcgacaatcaaattcaaaatatattaattaaactgtaatacttaataagagacaaataacagccaagaatcaaatactgggtttttaatcaaaagatctctctacatgcacccaaattcattatttaaatttactatactacagacagaatatacgaacccagattaagtagtcagacgcttttccgctttattgagtatatagccttacatattttctgcccataatttctggatttaaaataaacaaaaatggttactttgtagttatgaaaaaaggcttttccaaaatgcgaaatacgtgttatttaaggttaatcaacaaaacgcatatccatatgggtagttggacaaaacttcaatcgat

(19) United States
(12) Patent Application Publication
(10) Pub. No.: US 2014/0178933 A1
(43) Pub. Date: Jun. 26, 2014

(54) ENHANCED HETEROLOGOUS PROTEIN PRODUCTION IN KLUYVEROMYCES MARXIANUS

(71) Applicants: Hyun Min Koo, Seoul (KR); Byung Jo Yu, Hwaseong-si (KR); Ki Sung Lee, Suwon-si (KR); Jae Chan Park, Yongin-si (KR); Dae Hyuk Kweon, Suwon-si (KR); Sung Minm Park, Yongin-si (KR)

(72) Inventors: Hyun Min Koo, Seoul (KR); Byung Jo Yu, Hwaseong-si (KR); Ki Sung Lee, Suwon-si (KR); Jae Chan Park, Yongin-si (KR); Dae Hyuk Kweon, Suwon-si (KR); Sung Minm Park, Yongin-si (KR)

(73) Assignees: RESEARCH & BUSINESS FOUNDATION SUNGKYUNKWAN UNIVERSITY, Suwon-si, Gyeonggi-do (KR); Samsung Electronics Co., Ltd., Suwon-si, Gyeonggi-do (KR)

(21) Appl. No.: 14/114,886

(22) PCT Filed: Apr. 30, 2012
(86) PCT No.: PCT/KR2012/003379

§ 371 Date: Mar. 12, 2014

(30) Foreign Application Priority Data
Jun. 24, 2011 (KR) 10-2011-0061677

(51) Int. Cl. C12N 15/81; C12P 21/00

(52) U.S. Cl. CPC C12N 15/815; C12P 21/00
USPC 435/69.3; 435/320.1; 435/252.33; 435/254.2; 435/203; 435/224; 435/198; 435/189; 435/209; 435/197; 435/196; 435/192; 435/190; 435/233; 435/193; 435/200; 435/69.4; 435/69.1; 435/69.6

(57) ABSTRACT

An expression vector which is capable of overexpressing a protein of interest in a host cell, a host cell comprising the expression vector, and a method of producing a protein of interest are provided.

### TECHNICAL FIELD

This disclosure relates to an expression vector which is capable of overexpressing gene encoding a protein of interest in a host cell, a host cell comprising the expression vector, a method of expressing a gene product and a method of producing the protein of interest.

### BACKGROUND ART

With globally increasing concern about the exhaustion of resources and pollution of the environment by overuse of fossil fuels, the production of ethanol using microorganisms is being considered.

Currently, most ethanol is produced from feedstocks such as corn and cane sugar using strains of Saccharomyces cerevisiae (S. cerevisiae). However, the temperature suitable for growing conventional strains of S. cerevisiae should not be higher than a temperature of 35° C., and the ability of S. cerevisiae to utilize a carbon source including a pentose is low, thereby incurring a higher cost in producing ethanol.

Recently, strains of Kluyveromyces are being considered as viable alternatives to Saccharomyces cerevisiae. Kluyveromyces marxianus and Kluyveromyces Lactis are classified as GRAS (“Genenerally Recognized As Safe”) microorganisms, and may therefore be used with the same security as Saccharomyces cerevisiae.

K. marxianus is reported to grow at a temperature of 47° C., 49° C., and even 52° C., and the ability of K. marxianus to utilize a pentose such as xylose and arabinose as well as a polysaccharide such as lactose, inuline and celobiose is outstanding.

### DISCLOSURE OF INVENTION

### Technical Problem

However, K. marxianus has been studied insufficiently compared to many other microorganisms. Thus it is desirable to develop a promoter and an expression system which can express heterologous genes in K. marxianus at a high level, i.e., to permit genetic engineering in K. marxianus, for example, to achieve higher levels of production of a protein of interest.

### Solution to Problem

According to an aspect, an expression vector is disclosed. In an embodiment, the expression vector includes a replication origin, a promoter selected from the group consisting of CYC promoter, TEF promoter, GPD promoter and ADH promoter, and a terminator, wherein the expression vector is capable of overexpressing a gene encoding a protein of interest in a host cell.

According to another aspect, a host cell comprising the above expression vector and being capable of overexpressing a gene encoding a protein of interest is disclosed. In an embodiment, the host cell is a K marxianus cell.

According to another aspect, a method of producing a protein of interest is disclosed. In an embodiment, the method comprises culturing the host cell under suitable conditions for the expression of a gene encoding a protein of interest, and recovering a protein of interest.

### Advantageous Effects of Invention

The host cell may produce protein of interest at a higher level than the precursor host cell.

### BRIEF DESCRIPTION OF DRAWINGS

The above and other aspects of this disclosure will become more readily apparent by describing in further detail non-limiting exemplary embodiments thereof with reference to the accompanying drawings, in which:

FIG. 1 is a diagram depicting the plasmids pKM URA3 and pKM ΔURA3.

FIG. 2 presents photographic images a diagram showing image data of confirming growth of the K. marxianus uracil auxotroph in minimal media only in the presence of uracil.

FIG. 3 is a diagram depicting pKM316 according to Example 1.

FIG. 4 is a diagram depicting pJSKM316-CYC according to Example 2.

FIG. 5 is a diagram depicting pJSKM316-TEF according to Example 2.

FIG. 6 is a diagram depicting pJSKM316-GPD according to Example 2.

FIG. 7 is a diagram depicting pJSKM316-ADH according to Example 2.

FIG. 8 is a diagram depicting pJSKM316-CYC yEGFP CYC according to Example 3.

FIG. 9 is a diagram depicting pJSKM316-TEF yEGFP CYC according to Example 3.

FIG. 10 is a diagram depicting pJSKM316-GPD yEGFP CYC according to Example 3.

FIG. 11 is a diagram depicting pJSKM316-ADH yEGFP CYC according to Example 3.

FIG. 12 are graphs showing expression level of yEGFP using FACS.

FIG. 13 are graphs showing expression level of yEGFP using RT-PCR.

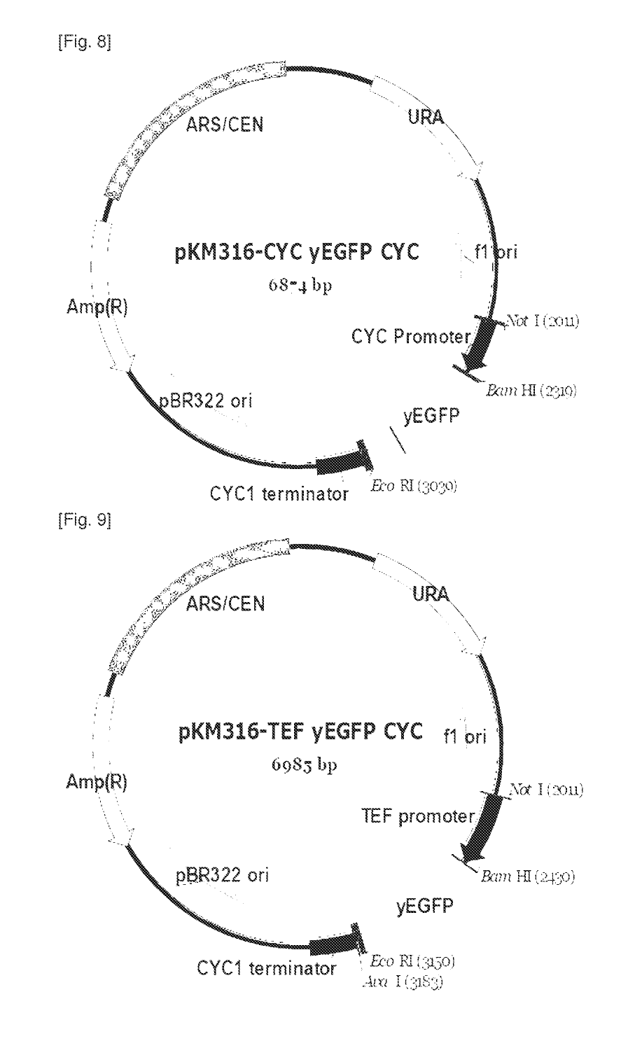

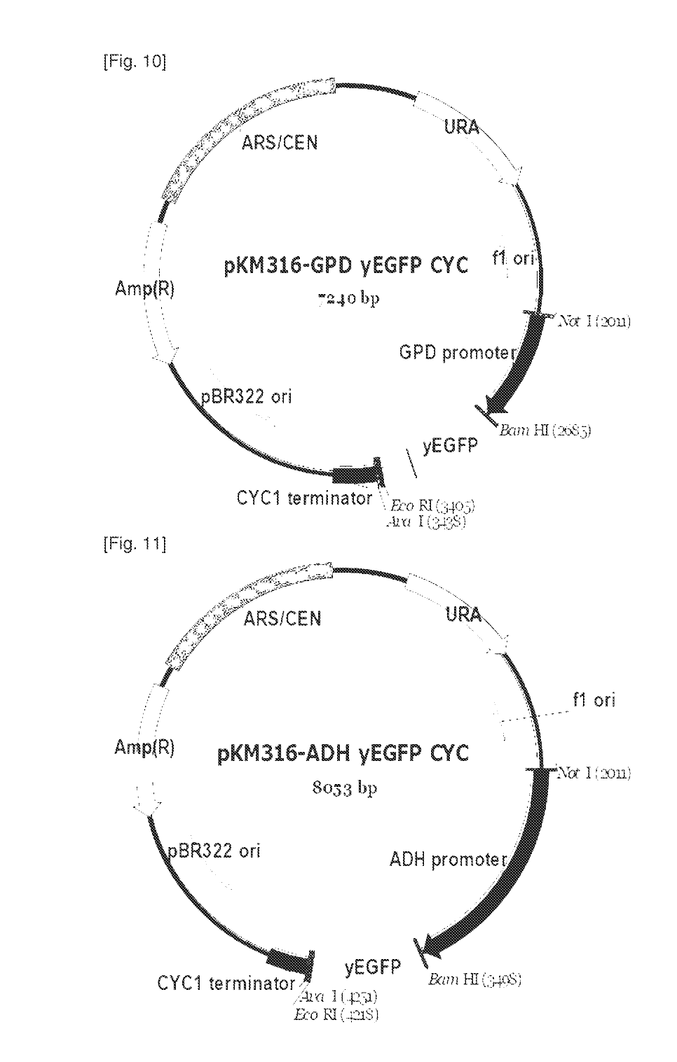

The expression vectors comprising each of the plasmids in FIGS. 8 to 11 are deposited, respectively. pJSKM316-CYC yEGFP CYC in FIG. 8 is deposited under the accession number KCTC11944BP, pJSKM316-TEF yEGFP CYC in FIG. 9 is deposited under the accession number KCTC11946BP, pJSKM316-GPD yEGFP CYC in FIG. 10 is deposited under the accession number KCTC11945BP, and pJSKM316-ADH yEGFP CYC in FIG. 11 is deposited under the accession number KCTC11943BP.

### BEST MODE FOR CARRYING OUT THE INVENTION

Unless otherwise indicated, the practice of the disclosure involves conventional techniques commonly used in molecular biology, microbiology, protein purification, protein engineering, protein and DNA sequencing, and recombinant DNA fields, which are within the skill of the art. Such techniques are known to those of skill in the art and are described in numerous standard texts and reference works. All patents, patent applications, articles and publications mentioned herein, both supra and infra, are hereby expressly incorporated herein by reference.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art.

As used herein, the singular terms “a”, “an,” and “the” include the plural reference unless the context clearly indicates otherwise. Unless otherwise indicated, nucleic acids are written left to right in 5′ to 3′ orientation and amino acid sequences are written left to right in amino to carboxyl orientation, respectively.

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The headings provided herein are not limitations of the various aspects or embodiments of the invention which can be had by reference to the specification as a whole.

### Promoter

According to an embodiment, a promoter which is an isolated polynucleotide capable of overexpressing a gene encoding a protein of interest is disclosed.

As used herein, the term “isolated” refers to a nucleic acid, an amino acid or other component that is removed from components with which it is naturally associated.

As used interchangeably herein, the terms “polynucleotide” and “nucleic acid” refer to a polymeric form of nucleotides of any length. These terms include, but are not limited to, a single-stranded DNA (“deoxyribonucleic acid”), double-stranded DNA, genomic DNA, cDNA, or a polymer comprising purine and pyrimidine bases, or other natural, chemically-modified, biochemically-modified, non-natural or derivatized nucleotide bases. Non-limiting examples of polynucleotides include genes, gene fragments, chromosomal fragments, ESTs, exons, introns, mRNA, tRNA, rRNA, ribozymes, cDNA, recombinant polynucleotides, branched polynucleotides, plasmids, vectors, isolated DNA of any sequence, isolated RNA (“ribonucleic acid”) of any sequence, nucleic acid probes, and primers. It will be understood that, as a result of the degeneracy of the genetic code, a multitude of nucleotide sequences encoding a given protein may be produced.

As used herein, the term “protein of interest” refers to a protein or a polypeptide that is produced by a host cell. Protein of interest is generally a protein that is commercially significant. The protein of interest may be either homologous or heterologous to the host cell. The term “heterologous protein” refers to a protein or a polypeptide that does not naturally occur in a host cell. The gene encoding the protein may be a naturally-occurring gene, a mutated or a synthetic gene. The term “homologous protein” refers to a protein or a polypeptide native or naturally occurring in a host cell. The homologous protein may be a native protein produced by other organisms.

As used herein, the term “operably linked” indicates that elements are arranged to perform the general functions of the elements. A nucleic acid is said to be “operably linked” when it is placed into a functional relationship with another nucleic acid sequence. For example, a polynucleotide promoter sequence is operably linked to a polynucleotide encoding a polypeptide if it affects the transcription of the sequence. The term “operably linked” may mean that the polynucleotide sequences being linked are contiguous. Linking may be accomplished by ligation at convenient restriction sites. If such sites do not exist, synthetic oligonucleotide adaptors or linkers may be used in accordance with conventional practice.

As used herein, the term “overexpression” refers to a process by which a gene comprising a sequence that encodes a polypeptide is artificially expressed in a modified cell to produce a level of expression of the transcript or the encoded polypeptide that exceeds the level of expression of the transcript or the encoded polypeptide in the unmodified precursor cell. Thus, while the term is typically used with respect to a gene, the term “overexpression” may also be used with a respect to an encoded protein to refer to the increased level of the protein resulting from the overexpression of its encoding gene. The overexpression of a gene encoding a protein may be achieved by various methods known in the art, e.g., by increasing the number of copies of the gene that encodes the protein, or by increasing the binding strength of the promoter region or the ribosome binding site in such a way as to increase the transcription or the translation of the gene that encodes the protein.

As used herein, the term “promoter” refers to a nucleic acid sequence that functions to drive or effect transcription of a downstream gene. In an embodiment, the promoter is functional in Kluyveromyces. In some embodiments, the promoter may be any promoter that drives expression of a gene encoding a protein of interest. A promoter may be any nucleic acid sequence which shows transcriptional activity in the host cell of choice and includes mutant, truncated and hybrid promoters, and may be obtained from genes encoding extracellular or intracellular polypeptides either homologous or heterologous to the host cell. The promoter sequence may be native or foreign to the host cell.

The protein of interest may be an enzyme. The enzyme may be amylolytic enzymes, proteolytic enzymes, cellulytic enzymes, oxidoreductase enzymes and plant wall degrading enzymes. For example, the enzyme may include, but is not limited to, amylases, proteases, xylanases, lipases, laccases, phenol oxidases, oxidases, cutinases, cellulases, hemicellulases, esterases, peroxidases, catalases, glucose oxidases, phytases, pectinases, glucosidases, isomerases, transferases, galactosidases and chitinases. Also, the protein of interest may be a hormone, cytokine, growth factor, receptor, vaccine, antibody, or the like.

It is not intended that the protein of interest be limited to any particular protein.

The promoter may be, but is not limited to, CYC (“cytochrome-c oxidase”), TEF (“translation elongation factor 1α”), GPD (“glyceraldehyde-3-phosphate dehydrogenase”), ADH (“alcohol dehydrogenase”), PHO5, TRP1, GAL1, GAL10, hexokinase, pyruvate decarboxylase, phosphofructokinase, triose phosphate isomerase, phosphoglucose isomerase, glucokinase, a-mating factor pheromone, GUT2, nmt, fbp1, AOX1, AOX2, MOX1, FMD1 and PGK1. In an embodiment, the promoter is functional in Kluyveromyces, specifically in K. marxianus.

In an exemplary embodiment, CYC promoter, TEL promoter, GPD promoter, or ADH promoter is used.

The CYC promoter may include SEQ ID NO: 1, or at least about 70%, at least about 75%, at least about 80%, at least about 85%, at least about 90%, at least about 92%, at least about 95%, at least about 97%, at least about 98% or at least about 99% sequence homology to the SEQ ID NO: 1.

SEQ ID NO: 1
atttggcgag cgttggttgg tggatcaagc ccacgcgtag
gcaatcctc gagcagatcc gccaggcgtg tatatatagc
gtggatggcc aggcaacttt agtgctgaca catacaggca
tatatatatg tgtgcgacga cacatgatc atatggcatg
catgtgctc tgtatgtata taaaactctt gttttcttct
tttctctaaa tattctttcc ttatacatta ggacctttg
cagcataaat tactatactt ctatagacac gcaaacacaa
atacacacac taa

The TEF promoter may include SEQ ID NO: 2, or at least about 70%, at least about 75%, at least about 80%, at least about 85%, at least about 90%, at least about 92%, at least about 95%, at least about 97%, at least about 98% or at least about 99% sequence homology to the SEQ ID NO: 2.

SEQ ID NO: 2
atagcttcaa aatgtttcta ctcctttttt actcttccag
attttctcgg actccgcgca tcgccgtacc acttcaaaac
acccaagcac agcatactaa atttcccctc tttcttcctc
tagggtgt cgttaattac ccgtactaaa ggtttggaaa
agaaaaaaga gaccgcctcg tttctttttc ttcgtcgaaa
aaggcaataa aaatttttat cacgtttctt tttcttgaaa
attttttttt tgattttttt ctctttcgat gacctcccat
tgatatttaa gttaataaac ggtcttcaat ttctcaagtt
tcagtttcat ttttcttgtt ctattacaac tttttttact
tcttgctcat tagaaagaaa gcatagcaat ctaatctaag ttt

The GPD promoter may include SEQ ID NO: 3, or at least about 70%, at least about 75%, at least about 80%, at least about 85%, at least about 90%, at least about 92%, at least about 95%, at least about 97%, at least about 98% or at least about 99% sequence homology to the SEQ 1D NO: 3.

SEQ ID NO: 3
agtttatcattatcaatactcgccatttcaaagaatacgtaaataattaa
tagtagtgattttcctaactttatttagtcaaaaaattagccttttaatt
ctgctgtaacccgtacatgcccaaaatagggggcgggttacacagaatat
ataacatcgtaggtgtctgggtgaacagtttattcctggcatccactaaa
tataatggagcccgctttttaagctggcatccagaaaaaaaaagaatccc
agcaccaaaatattgttttcttcaccaaccatcagttcataggtccattc
tcttagcgcaactacagagaacaggggcacaaacaggcaaaaaacgggca
caacctcaatggagtgatgcaacctgcctggagtaaatgatgacacaagg
caattgacccacgcatgtatctatctcattttcttacaccttctattacc
ttctgctctctctgatttggaaaaagctgaaaaaaaaggttgaaaccagt
tccctgaaattattcccctacttgactaataagtatataaagacggtagg
tattgattgtaattctgtaaatctatttcttaaacttcttaaattctact
tttatagttagtctttttttagttttaaaacaccagaacttagtttcgac
ggat

The ADH promoter may include SEQ ID NO: 4, or at least about 70%, at least about 75%, at least about 80%, at least about 85%, at least about 90%, at least about 92%, at least about 95%, at least about 97%, at least about 98% or at least about 99% sequence homology to the SEQ ID NO: 4.

SEQ ID NO: 4
gccgggatcg aagaaatgat ggtaaatgaa ataggaaatc
aaggagcatg aaggcaaaa gacaaatata agggtcgaac
gaaaaataaa gtgaaaagtg ttgatatgat gtatttggct
ttgcggcgcc gaaaaaacga gtttacgcaa ttgcacaatc
atgctgactc tgtggcggac ccgcgctctt gccggcccgg
cgataacgct gggcgtgagg ctgtgcccgg cggagttttt
tgcgcctgca ttttccaagg tttaccctgc gctaaggggc
gagattggag aagcaataag aatgccggtt ggggttgcga
tgatgacgac cacgacaact ggtgtcatta tt-taagttgc
cgaaagaacc tgagtgcatt tgcaacatga gtatactagaa
gaatgagcca agacttgcga gacgcgagtt tgccggtggt
gcgaacaata gagcgaccat gaccttgaag gtgagacgcg
cataaccgct agagtacttt gaagaggaaa cagcaatagg
gttgctacca gtataaatag acaggtacat acaacactgg
aaatggttgt ctgtttgagt acgctttcaa ttcatttggg
tgtgcacttt attatgttac aatatggaag ggaactttac
acttctcctat gcacatatat taattaaagt ccaatgctag
tagagaaggg gggtaacacc cctccgcgct cttttccgat
ttttttctaa accgtggaat atttcggatat ccttttgttg
tttccgggtg tacaatatgg acttcctctt ttctggcaac
caaacccata catcgggatt cctataatac cttcgttggt
ctccctaaca tgtaggtggc ggaggggaga tatacaatag
aacagatacc agacaagaca taatgggcta aacaagacta
caccaattac actgcctcat tgatggtggt acataacgaa
ctaatactgt agccctaga cttgatagc catcatcat
atcgaagttt cactaccctt tttccatttg ccatctattg
aagtaataat aggcgcatgc aacttctttt cttttttttt
cttttctctc tcccccgttg ttgtctcacca tatccgcaat
gacaaaaaaa tgatggaagaca ctaaaggaaa aaattaacga
caaagacagc accaacagat gtcgttgttc cagagctgat
gaggggtatc tcgaagcaca cgaaactttt tccttccttc
attcacgcaca ctactctcta atgagcaacg gtatacggcc
ttccttccag ttacttgaat ttgaaataaa aaaaagtttg
ctgtcttgct atcaagtataa atagacctgc aattattaat
cttttgtttc ctcgtcattgt tctcgttccc tttcttcctt
gtttcttttt ctgcacaata tttcaagcta taccaagcat
acaatcaact ccaagctggc cgc

As used herein, the term “homology” refers to sequence similarity or identity. This homology may be determined using standard techniques known in the art (See e.g., Smith and Waterman, Adv. Appl. Math., 2:482 [1981]; Needleman and Wunsch, J. Mol. Biol., 48:443 [1970]; Pearson and Lipman, Proc. Natl. Acad. Sci. USA 85:2444 [1988]; programs such as GAP, BESTFIT, FASTA, and TFASTA in the Wisconsin Genetics Software Package (Genetics Computer Group, Madison, Wis.); and Devereux et al., Nucl. Acid Res., 12:387-395 [ 19841).

Expression Vector

According to another embodiment, an expression vector which is a polynucleotide comprising a gene encoding a protein of interest, a promoter and a terminator is provided. In an embodiment, the expression vector is suitable for expression of the gene in K. marxianus. In an embodiment, the gene is a gene encoding a protein of interest.

As used herein, the term “expression vector” refers to a DNA construct containing a DNA sequence that is operably linked to a suitable control sequence capable of effecting the expression of the DNA in a suitable host. The vector may be a plasmid, a phage particle, or simply a potential genomic insert. Once transformed into a suitable host, the vector replicates and functions independently of the host genome, or integrates into the genome itself. As used herein, the terms “plasmid,” “expression plasmid,” and “vector” are often used interchangeably as a plasmid is among the most commonly used forms of vector at present.

However, it is intended to include such other forms of expression vectors that serve equivalent functions and which are, or become, known in the art. For example, the vector may be a cloning vector, an expression vector, a shuttle vector, a plasmids, a phage or virus particle, a DNA construct, or a cassette. As used herein, the term “plasmid” refers to a circular doublestranded DNA construct used as a cloning vector, and which forms an extrachromosomal self replicating genetic element in many bacteria and some eukaryotes. The plasmid may be a multicopy plasmid that can integrate into the genome of the host cell by homologous recombination.

As known to those skilled in the art, to increase the expression level of a gene introduced to a host cell, the gene should be operably linked to expression control sequences for the control of transcription and translation which function in the selected expression host. For example, the expression control sequences and the gene are included in one expression vector together with a selection marker and a replication origin. When the expression host is a eukaryotic cell, the expression vector should further include an expression marker useful in the eukaryotic expression host.

As used herein, the term “gene” refers to a chromosomal segment of DNA involved in producing a polypeptide chain that may or may not include regions preceding and following the coding regions, for example, 5′ untranslated (“5′ UTR”) or leader sequences and 3′ untranslated (“3′ UTR”) or trailer sequences, as well as intervening sequence (introns) between individual coding segments (exons).

As used herein, the term “terminator” refers to a nucleic acid sequence that functions to drive or effect termination of transcription. In an embodiment, the terminator is functional in Kluyveromyces.

In the embodiment, the gene may encode a protein that has commercial significance such as an enzyme, hormone, cytokine, growth factor, receptor, vaccine, antibody, or the like. The gene encoding the protein may be a naturally occurring gene, a mutated gene, or a synthetic gene. It is not intended that the gene be limited to any particular gene.

In an embodiment, the promoter is as defined above.

In one embodiment, the terminator may be, but is not limited to, CYC1 (“cytochrome c transcription”) terminator or GAL1 terminator. In an exemplary embodiment, CYC1 terminator is used.

The CYC1 terminator may include SEQ ID NO: 5, or at least about 70%, at least about 75%, at least about 80%, at least about 85%, at least about 90%, at least about 92%, at least about 95%, at least about 97%, at least about 98% or at least about 99% sequence homology to the SEQ ID NO: 5.

SEQ ID NO: 5

tcatgtaatt agttatgtca cgcttacatt cacgccctcc ccccacatcc gctctaacc gaaaaggaag gagttagaca acctgaagtc taggtcccta tttatttttt tatagttatg ttagtattaa gaacgttatt tatatttca aatttttct tttttttctg tacagacgc gtgtacgca tgtaacattat actgaaaacc ttgcttgaga aggttttggg acgctcgaag gctttaattt gcggcc

In one embodiment, the expression vector may further comprise a selectable marker.

As used herein, the term “selectable marker” refers to a nucleotide sequence which is capable of expression in the host cells and where expression of the selectable marker confers to cells containing the expressed gene the ability to grow in the presence of a corresponding selective agent or in the absence of an essential nutrient. For example, the selectable marker may be, but is not limited to, resistance genes to antimicrobials such as kanamycin, erythromycin, actinomycin, chloramphenicol and tetracycline, or essential nutrient biosynthetic gene such as URA3, LEU2, TRP1 and HTS3. That is, selectable markers are genes that confer antimicrobial resistance or alter nutrient requirements of the host cell to allow cells containing the exogenous DNA to be distinguished from cells that have not received any exogenous sequence during the transformation.

In an exemplary embodiment, URA3 is used as a selectable marker gene.

In an embodiment, the expression vector may further comprise a replication origin.

As used herein, the term “replication origin” refers to a nucleotide sequence at which replication or amplification of a plasmid begins in a host cell. The replication origin may include an autonomous replication sequence (“ARS”), and the ARS may be stabilized by a centromeric sequence (“CEN”). In an embodiment, ARS/CEN is from a Kluyveromyces. In an exemplary embodiment, ARS/CEN from K. marxianus is used.

The ARS/CEN replication origin may include SEQ ID NO: 6, or at least about 70%, at least about 75%, at least about 80%, at least about 85%, at least about 90%, at least about 92%, at least about 95%, at least about 97%, at least about 98% or at least about 99% sequence homology to the SEP ID NO: 6.

SEQ ID NO: 6
gagctccttt catttctgat aaaagtaaga ttactccatt
tatcttttca ccaacatat tcatagttga aagttatcct tc-
taagtacg tatacaatat taattaaacg taaaaacaaa
actgactgta aaaatgtgta aaaaaaaaat atcaaattc
atagcagttt caaggaatga aaactattat gatctggtca
cgtgtatata aattattaat tttaaaccca tataatttat
tattttttta ttctaaagt ttaaagtaat tttagtagt
attttatatt ttgaataaat atactttaaa tttttatttt
tatattttat tacttttaaa aataatgttt ttatttaaaa
caaaattata agttaaaaag ttgttccgaa agtaaaatat
attttatagt ttttacaaaa ataaattatt tttaacgtat
tttttttaat tatatttttg tatgtgatta tatccacagg
tattatgctg aatttagctg tttcagttta ccagtgtgat
agtatgattt tttttgcctct caaaagctatt tttttagaag
cttcgtctta gaaataggtg gtgtataaat tgcggttgac
ttttaactat atatcatttt cgatttattt attacataga
gaggtgcttt taatttttta atttttattt tcaataattt
taaaagtggg tacttttaaa ttggaacaaa gtgaaaaata
tctgttatac gt-gcaactga attttactga ccttaaagga
ctatctcaat cctggttcag aaatccttgaa atgattgata
tgttggtgg attttctctg attttcaaac aagaggtat
tttatttcat atttattata ttttttacat ttattttata
tttttttatt gtttggaagg gaaagcgaca atcaaattca
aaatatatta attaaactgt aatacttaat aagagacaaa
taacagccaa gaatcaaat actgggtttt taatcaaaag
atctctctac atgcacccaa attcattatt taaatttact
atactacaga cagaatatac gaacccagat taagtagtca
gacgcttttc cgctttattg agtatatagc cttacatatt
ttctgcccat aatttctgga tt-taaaataa acaaaaatgg
ttactttgta gttatgaaaa aaggcttttc caaaatgcga
aatacgtgtt atttaaggtt aatcaacaaa acgcatatcc
atatgggtag ttggacaaaa cttcaatcga t

Host Cell

In another embodiment, a host cell comprising the above promoters is provided. That is, the host cell may include a promoter selected from the group consisting of CYC promoter, TEF promoter, GPD promoter and ADH promoter. Also, the host cell may further include a CYC1 terminator or ARS/CEN replication origin, CYC1 terminator and ARS/CEN replication origin.

As used herein, the term “host cell” refers to a suitable cell that serves as a host for an expression vector. A suitable host cell may be a naturally occurring or wildtype host cell, or it may be an altered host cell. A “wildtype host cell” is a host cell that has not been genetically altered using recombinant methods.

As used herein, the term “altered host cell” refers to a genetically engineered host cell wherein a gene is expressed at an altered level of expression compared to the level of expression of the same gene in an unaltered or wildtype host cell grown under essentially the same growth conditions. In an embodiment, an altered host cell is one in which the gene encoding a protein of interest is expressed or produced at a level of expression or production that is higher than the level of expression or production of the gene in the unaltered or wildtype host cell grown under essentially the same growth conditions. A “modified host cell” herein refer to a wildtype or altered host cell that has been genetically engineered to overproduce a protein of interest. A modified host cell is capable of producing a protein of interest at a greater level than its wildtype or altered parent host cell.

As used herein, the term “parent” or “precursor” cell refers to a cell from which a modified or an altered host cell is derived. The parent or precursor cell of a modified host cell can be a wildtype cell or an altered cell.

As used herein, the term “recombinant” refers to a polynucleotide or a polypeptide which is not endogenous to a host cell.

In an embodiment, the host cell may be, but is not limited to, a cell from the genus Kluyveromyces or the genus Escherichia. For example, the genus Kluyveromyces may be, but is not limited to, K marxianus, K. fragilis, K. lactis, K. bulgaricus, and K. thermotolerans. In an exemplary embodiment, a K. marxianus cell or an E. coli cell is used.

Method of Producing Protein of Interest

According to another embodiment, a method of producing a protein of interest is provided. In an embodiment, the method comprises culturing a host cell under conditions which are suitable for expression of a gene encoding the protein of interest, and recovering the protein of interest. According to the embodiment, the level of expression of the gene encoding the protein of interest increases in the host cell, so that production of the protein of interest may be enhanced. In some embodiments, the method further comprises introducing into a host cell an expression vector for overexpressing a gene encoding a protein of interest.

As used herein, the term “introduced” refers to any method suitable for transferring the nucleic acid sequence into the cell. Such a method for introduction may be, but is not limited to, protoplast fusion, transfection, transformation, conjugation, and transduction (See e.g., Ferrari et al., “Genetics,” in Hardwood et al., (eds.), Bacillus, Plenum Publishing Corp., pages 5772, [1989]).

As used herein, the terms “transformed” and “stably transformed” refer to a cell that has a nonnative heterologous polynucleotide sequence integrated into its genome or has the heterologous polynucleotide sequence present as an episomal plasmid that is maintained for at least two generations.

As used herein, the term “early expression or “early production” indicates that the expression of a gene or production of a protein of interest occurs in a host cell in a time that is earlier than that in which the gene is normally expressed or protein of interest is normally produced by the precursor/parent host.

As used herein, the term “enhanced” refers to improved production of protein of interests. That is, the “enhanced” production is improved as compared to the normal levels of production by the unmodified wild type or altered parent host.

In an embodiment, the introduction of a polynucleotide into a host cell may be conducted by transforming the polynucleotide into the host cell. In some embodiments, the polynucleotide, e.g., a plasmid, can be grown in and isolated from an intervening microorganism, e.g., an E. coli. Transformation may be achieved by any one of various means including electroporation, microinjection, biolistics (or particle bombardment-mediated delivery), or agrobacteriummediated transformation.

In an embodiment, modified host cells may be cultured under suitable conditions for the expression and recovery of protein of interest from the cell culture. Specifically, the protein of interest produced by the modified host cells is recovered from the culture medium by conventional procedures, including, but not limited to separating the host cells from the medium by centrifugation or filtration, precipitating the proteinous components of the supernatant or filtrate by means of a salt such as ammonium sulfate, chromatographic purification such as ion exchange, gel filtration, affinity, etc. . . . It is not intended that the culture condition be limited to any particular method.

In an embodiment, altered host cells may be cultured under suitable conditions for expression of a protein of interest. It is not intended that the culture conditions be limited to any particular conditions. In some embodiments, the protein of interest is recovered from the cell culture. Specifically, the protein of interest produced by the altered host cells is recovered from the culture medium by conventional procedures, such as separating the host cells from the medium by centrifugation or filtration, precipitating protein components of the supernatant or filtrate by means of a salt such as ammonium sulfate, or chromatographic purification, for example ion exchange, gel filtration, or affinity, chromatography.

The medium used to culture the cells comprises any conventional suitable medium known in the art for growing the host cells, such as minimal or complex media containing appropriate supplements. Suitable media are available from commercial suppliers or may be prepared according to published recipes (e.g., in catalogues of the American Type Culture Collection).

The host cells may be cultured under batch, fedbatch or continuous fermentation conditions. Classical batch fermentation methods use a closed system, in which the culture medium is made prior to the beginning of the fermentation run, the medium is inoculated with the desired organisms, and fermentation occurs without subsequent addition of any components to the medium. In certain cases, the pH or oxygen content of the growth medium is altered during batch methods, but the content carbon source content is not altered. The metabolites and cell biomass of the batch system change constantly up to the time the fermentation is stopped. In a batch system, cell growth usually progresses through a static lag phase to a high growth log phase and finally to a stationary phase where the growth rate is diminished or halted. If untreated, cells in the stationary phase eventually die. In general, cells in log phase produce the most protein.

A variation on the standard batch fermentation is a “fedbatch fermentation” system. In fedbatch fermentation system, nutrients (e.g., a carbon source, nitrogen source, O2, and typically, other nutrients) are only added when their concentration in culture falls below a threshold. Fedbatch systems are useful when catabolite repression is apt to inhibit the metabolism of the cells and where it is desirable to have limited amounts of nutrients in the medium. Actual nutrient concentration in fedbatch systems are estimated on the basis of the changes of measurable factors such as pH, dissolved oxygen and the partial pressure of waste gases such as CO₂. Batch and fedbatch fermentations are common and well known in the art.

Continuous fermentation is an open system in which a defined culture medium is added continuously to a bioreactor and an equal amount of conditioned medium is removed simultaneously for processing. Continuous fermentation generally maintains the cultures at a constant high density where cells are primarily in log phase growth. Continuous fermentation allows for the modulation of one factor or any number of factors that affect cell growth and/or end product concentration. For example, a limiting nutrient such as the carbon source or nitrogen source is maintained at a fixed rate and all other parameters are allowed to moderate. In other systems, a number of factors affecting growth are altered continuously while the cell concentration, measured by media turbidity, is kept constant. Continuous systems strive to maintain steady state growth conditions. Thus, cell loss due to medium being drawn off may be balanced against the cell growth rate in the fermentation. Methods of modulating nutrients and growth factors for continuous fermentation processes as well as techniques for maximizing the rate of product formation are known to those of skill in the art.

There are various assays known to those of ordinary skill in the art for detecting and measuring expression of a gene encoding a protein of interest in host cells. The assay may be, but is not limited to fluorescence activated cell sorting (“FACS”), real time-Polymerase Chain Reaction (“RTPCR”), enzyme linked immunosorbent assay (“ELISA”), radioimmunoassay (“RIA”), and fluorescence immunoassay (“FIA”). In an exemplary embodiment, FACS or RTPCR are used.

According to an embodiment, an altered host cell may produce the protein of interest at a greater level and in a shorter time than its precursor host cell. For example, the production of the protein of interest in the altered host cell may be about 20%, about 30% or about 50% greater than that in the precursor host cell. Also, the time for expressing the protein of interest in the altered host cell may be about ⅕, about ¼, about ⅓ or about ½ shorter than that in the precursor host cell.

Hereinafter, the invention will be described in further detail with respect to exemplary embodiments. However, it should be understood that the invention is not limited to these Examples and may be embodied in various modifications and changes.

### MODE FOR THE INVENTION

Strain and Plasmid

E. coli DH5α (F⁻ endA1 glnV44 thi-1 recA1 relA1 gyrA96 deoR nupGφ80dlacZΔM15Δ(lacZYA-argF)U169,hsdR17[r_{K}⁻ m_{K}⁺], λ⁻) (Invitrogen, Gaithersburg, Md.) is used for amplification of a plasmid. Kluyveromyces marxianus var. marxianus (KTCT 17555) is used as a yeast host cell for expression of a protein encoding a protein of interest. The integrating yeast-E. coli shuttle vector plasmid pRS306 (ATCC 77141) is used.

Medium and Method for Culturing

E. coli is inoculated in LB medium (1% bacto-trypton, 0.5% bacto-yeast extract, 1% NaCl) having ampicillin and kanamycin, and then cultured at a temperature of 37° C. A yeast host cell and a recombinant yeast are cultured in YPD medium (1% bacto-yeast extract, 2% bacto-pepton, 2% dextrose) at a temperature of 37° C. for 2 days. Minimal medium includes 0.17% yeast nitrogen base, 0.5% ammonium sulfate, 2% glucose or glycerol, 38.4 mg/l arginine, 57.6 mg/l isoleucine, 48 mg/l phenylalanine, 57.6% mg/l valine, 6 mg/lthreonine, 50 mg/l inositol, 40 mg/l tryptophan, 15 mg/l tyrosine, 60 mg/l leusine and 4 mg/l histidine.

### EXAMPLE 1

### Construction of K. marxianus Uracil Auxotroph

A K. marxianus Uracil auxotroph is prepared so that K. marxianus may use uracil as a selectable marker.

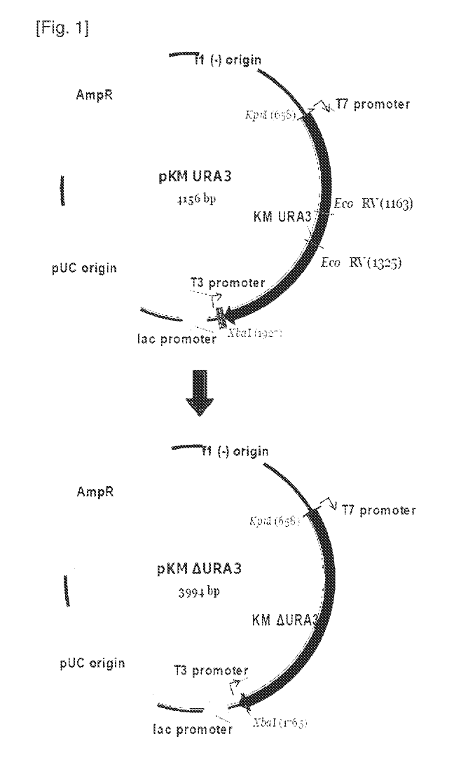

The K. marxianus Ura3(KmUra3) gene is digested with restriction enzymes KpnI and XbaI, and then ligated into the vector, pBluescript SK II(−) (Stratagene) which is digested with the same restriction enzymes to construct pKM URA3. This plasmid is digested with the restriction enzyme EcoRV, the ˜4 kbp EcoRV fragment that has the KmUra3 gene with a missing EcoRV fragment was isolated and re-ligated to construct pKM URA3. The plasmids pKM URA3 and pKM URA3 are shown in FIG. 1.

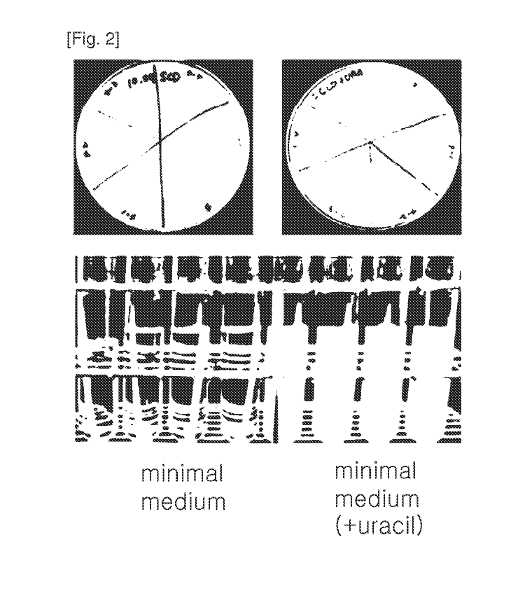

This plasmid has a non-functional URA3 gene (ΔURA3). The plasmid is digested with restriction enzymes KpnI and NotI, and transformed into K marxianus (KTCT 17555) by electroporation to construct a K. marxianus uracil auxotroph. The transformants are cultured in minimal medium and minimal medium with uracil. The growth patterns observed, shown in FIG. 2, show that uracil auxotrophs are successfully constructed.

### EXAMPLE 2

### Construction of Recombinant Expression Vectors

A recombinant vector for expressing a gene in K. marxianus is prepared.

The ARS/CEN replication origin from K marxianus is amplified by means of a polymerase chain reaction (PCR) at an optimal annealing temperature (TaOpt) of 53.2° C. using the following primers:

Forward(FW) primer:
5′-TTCAGACGTCGAGCTCCTTTCATTTCTGAT-3′
Backward(BW) primer:
5′-TTCAGACGTCATCGATTGAAGTTTTGTCCA-3′

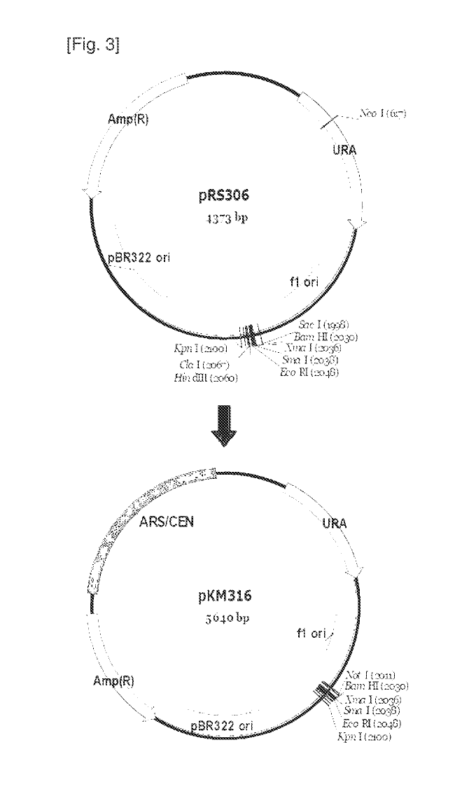

Next, the replication origin is digested with the restriction enzyme AatII, and then ligated into the plasmid pRS306 (ATCC 77141), digested with the same restriction enzyme to construct a K marxianus-E. coli shuttle vector, which is referred to as pKM316. The K. marxianus-E. coli shuttle vector is shown in FIG. 3.

A. pJSKM316 CYC

A CYC promoter from S. cerevisiae and a CYC terminator from K. marxianus are each amplified by means of PCR at TaOpt of 58.5° C.

Next, the amplified promoter and the terminator amplicons are digested with restriction enzymes NotI and KpnI, and then ligated into pKM316, digested with the same restriction enzymes to construct pJSKM316-CYC. The plasmid pJSKM316-CYC is shown in FIG. 4.

B. pJSKM316 TEF

A TEF promoter from S. cerevisiae and a CYC terminator from K. marxianus are amplified by means of PCR at TaOpt of 58.1° C.

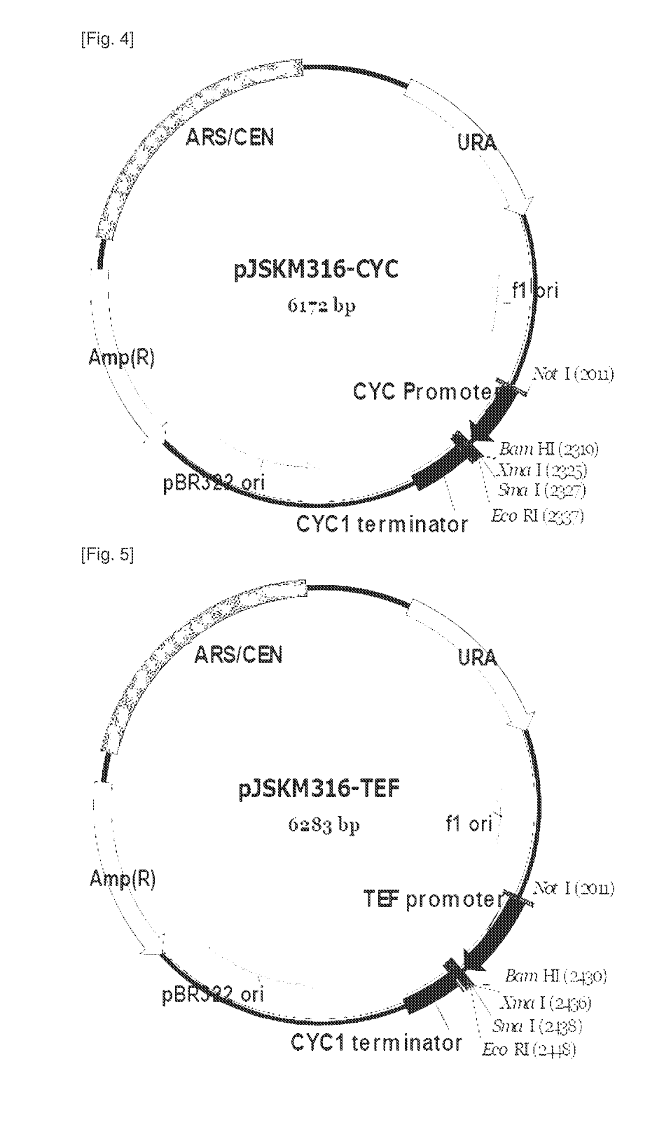

Next, the amplified promoter and the terminator are digested with restriction enzymes NotI and KpnI, and then ligated into pKM316 which is digested with the same restriction enzymes to construct pJSKM316-TEF. The plasmid pJSKM316-TEF is shown in FIG. 5.

C. pJSKM316 GPD

A GPD promoter from S. cerevisae and a CYC terminator from K. marxianus are amplified by means of PCR at TaOpt of 57.5° C.

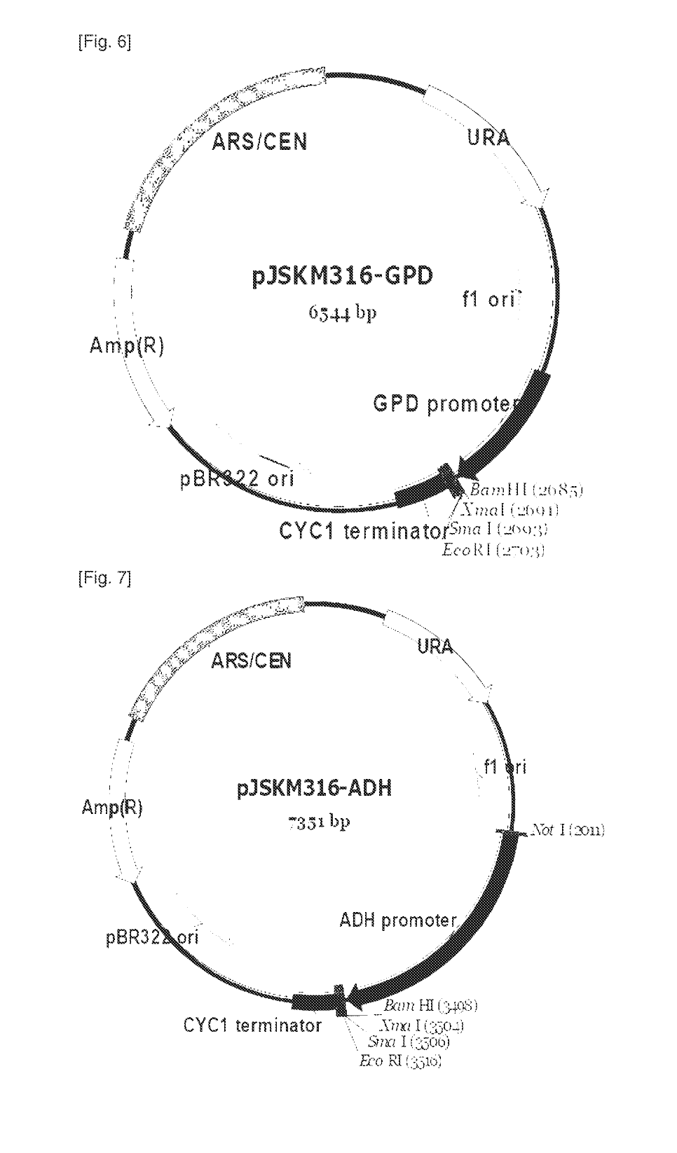

Next, the amplified promoter and the terminator are digested with restriction enzymes NotI and KpnI, and then ligated into pKM316 which is digested with the same restriction enzymes to construct pJSKM316-GPD. The plasmid pJSKM316-GPD is shown in FIG. 6.

D. pJSKM316 ADH

A ADH promoter from S. cerevisiae and a CYC terminator from K. marxianus are amplified by means of PCR at TaOpt of 57.5° C.

Next, the amplified promoter and the terminator are digested with restriction enzymes NotI and KpnI, and then ligated into pKM316 which is digested with the same restriction enzymes to construct pJSKM316-ADH. The plasmid pJSKM316-ADH is shown in FIG. 7.

### EXAMPLE 3

### Construction of Recombinant Expression Vector for Assaying Activity of Promoter

Yeast enhanced green fluorescent protein 3 (yEGFP) is used to evaluate the expression level of the constructed expression vectors. The yEGFP absorbs light from 395 nm to 470 nm 509 nm and emits green fluorescence at 509 nm.

A. pJSKM316 CYC yEGFP CYC

The yEGFP is digested with restriction enzymes NotI and KpnI, and then ligated into pJSKM316-CYC, digested with the same restriction enzymes to construct pJSKM316-CYC yEGFP CYC. The pJSKM316-CYC yEGFP CYC is shown in FIG. 8. The constructed vector has the yEGFP gene, under the control of the CYC promoter.

B. pJSKM316-TEF yEGFP CYC

pJSKM316-TEF yEGFP CYC is constructed by the same method as above, except that pJSKM316-TEF is used instead of pJSKM316-CYC. The pJSKM316-TEF yEGFP CYC is shown in FIG. 9. The constructed vector has the yEGFP gene, under the control of the TEF promoter.

C. pJSKM316-GPD yEGFP CYC

pJSKM316-GPD yEGFP CYC is constructed by the same method as above, except that pJSKM316-GPD is used instead of pJSKM316-CYC. The pJSKM316-GPD yEGFP CYC is shown in FIG. 10. The constructed vector has the yEGFP gene, under the control of the GPD promoter.

D. pJSKM316-ADH yEGFP CYC

pJSKM316-ADH yEGFP CYC is constructed by the same method as above, except that pJSKM316-ADH is used instead of pJSKM316-CYC. The pJSKM316-ADH yEGFP CYC is shown in FIG. 11. The constructed vector has the yEGFP gene, under the control of the ADH promoter.

Each of these constructed expression vectors is transformed into a K. marxianus uracil auxotroph of Example 1, respectively, and then the activities of the CYC promoter, the TEF promoter, the GPD promoter and the ADH promoter in the various transformed strains is measured.

### EXAMPLE 4

### Analysis of Expression Level of yEGFP Using FACS

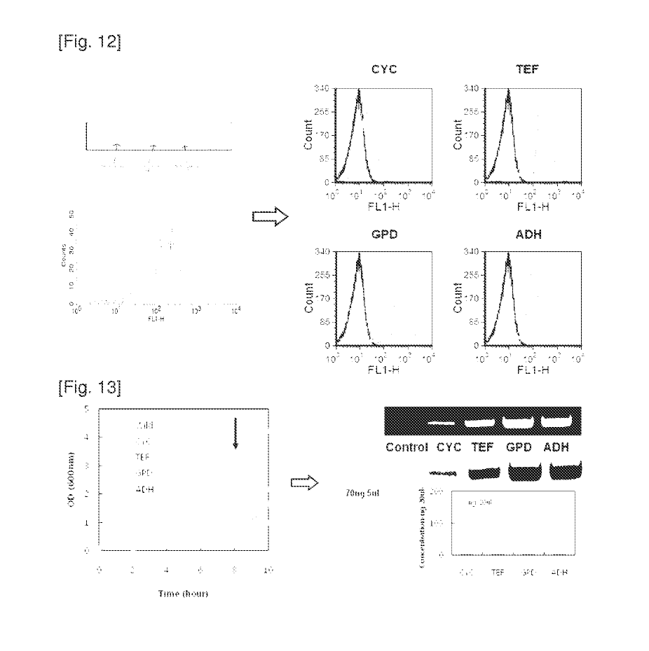

The expression level of yEGFP by the promoters in K marxianus is measured by fluorescence-activated cell sorting (FACS). The results are shown in FIG. 12.

Referring to FIG. 12, it can be seen that the expression level of yEGFP in the various transformed strains is high, and overexpression decreases in the order of the GPD promoter, the ADH promoter, the TEF promoter, and then the CYC promoter.

That is, it is verified that the expression level of yEGFP in K. marxianus including each of the GPD promoter, the ADH promoter, the TEF promoter, and the CYC promoter is about 20%, about 30%, about 40% or about 50% higher than that of the precursor host cell.

### EXAMPLE 5

### Analysis of Expression Level of yEGFP Using RT-PCR

The whole RNA of K. marxianus is isolated, and cDNA is synthesized using the RNA as a template, amplified by means of PCR. Then, RT-PCR (Reverse Transcriptase-Polymerase Chain Reaction) is performed using the specific primers for yEGFP target gene. The result is shown in Table 1 and FIG. 13.

TABLE 1

Promoter

| | CYC | TEF | GPD | ADH |
|---|---|---|---|---|
| | promoter | promoter | promoter | promoter |
| ng/20 ul | 104.6712 | 139.4863 | 168.5153 | 164.6932 |

Referring to Table 1 and FIG. 13, the expression level of yEGFP mRNA in the transformed strains is high, and overexpression decreases in the order of the GPD promoter, the ADH promoter, the TEF promoter, and then the CYC promoter.

The expression time of K. marxianus including each of the GPD promoter, the ADH promoter, the TEF promoter, and the CYC promoter is shown to be about ¼, about ⅓ or about ½ shorter than that of the precursor host cell.

While example embodiments have been disclosed herein, it should be understood that other variations may be possible. Such variations are not to be regarded as a departure from the spirit and scope of the invention, and all such modifications as would be obvious to one skilled in the art are intended to be included within the scope of the following claims.

### SEQUENCE LISTING FREE TEXT

<110> SAMSUNG ELECTRONICS CO., LTD.

<120> ENHANCED PROTEIN PRODUCTION IN KLUYVEROMYCES MARXIANUS.

<130> 2011-PP-148

<160> 6

<170> KopatentIn 2.0

<210> 1

<211> 289

<212> DNA

<213> Artificial Sequence

<220>

<223> Artificial Sequence

<400> 1.

atttggcgag cgttggttgg tggatcaagc ccacgcgtag gcaatcctcg agcagatccg 60

ccaggcgtgt atatatagcg tggatggcca ggcaacttta gtgctgacac atacaggcat 120

atatatatgt gtgcgacgac acatgatcat atggcatgca tgtgctctgt atgtatataa 180

1. An expression vector, comprising:
a replication origin permitting replication of the vector in a Kluyveromyces cell;
a promoter functional in Kluyveromyces selected from the group consisting of CYC promoter, TEF promoter, GPD promoter and ADH promoter; and
a terminator.

2. The expression vector of claim 1, wherein the CYC promoter comprises SEQ ID NO. 1 or at least 70% sequence homology to the SEQ ID NO. 1; the TEF promoter comprises SEQ ID NO. 2 or at least 70% sequence homology to the SEQ ID NO. 2; the GPD promoter comprises SEQ ID NO. 3 or at least 70% sequence homology to the SEQ ID NO. 3; and the ADH promoter comprises SEQ ID NO. 4 or at least 70% sequence homology to the SEQ ID NO. 4.

3. The expression vector of claim 1, wherein the replication origin is a Kluyveromyces marxianus ARS/CEN (autonomous replication sequence/centromeric) sequence.

4. The expression vector of claim 3, wherein the ARS/CEN replication origin comprises SEQ ID NO. 6 or at least 70% sequence homology to the SEQ ID NO. 6.

5. The expression vector of claim 1, wherein the terminator is a Kluyveromyces marxianus CYC1 (cytochrome-c oxidase) terminator.

6. The expression vector of claim 5, wherein the CYC1 terminator comprises SEQ ID NO. 5 or at least 70% sequence homology to the SEQ ID NO. 5.

7. The expression vector of claim 1, wherein the expression vector comprises a nucleic acid sequence encoding at least one selected from the group consisting of amylases, proteases, xylanases, lipases, laccases, phenol oxidases, oxidases, cutinases, cellulases, hemicellulases, esterases, peroxidases, catalases, glucose oxidases, phytases, pectinases, glucosidases, isomerases, transferases, galactosidases, chitinases, hormones, cytokines, growth factors, receptors, vaccines and antibodies.

8. The expression vector of claim 1, wherein the expression vector is selected from the group consisting of pJSKM316-ADH yEGFP CYC deposited under the accession number KCTC11943BP, pJSKM316-CYC yEGFP CYC deposited under the accession number KCTC11944BP, pJSKM316-GPD yEGFP CYC deposited under the accession number KCTC11945BP and pJSKM316-TEF yEGFP CYC deposited under the accession number KCTC11946BP.

9. (canceled)

10. A host cell comprising the expression vector of claims 1.

11. The host cell of claim 10, wherein the host cell is Kluyveromyces marxianus or Escherichia coli.

12. The host cell of claim 10, wherein the expression vector overexpresses a gene encoding a protein of interest that is heterologous to the host cell.

13. The host cell of claim 10, wherein the protein of interest is at least one selected from the group consisting of amylases, proteases, xylanases, lipases, laccases, phenol oxidases, oxidases, cutinases, cellulases, hemicellulases, esterases, peroxidases, catalases, glucose oxidases, phytases, pectinases, glucosidases, isomerases, transferases, galactosidases, chitinases, hormones, cytokines, growth factors, receptors, vaccines and antibodies.

14. A method of producing a protein of interest, comprising:
culturing the host cell of claim 13 under suitable conditions for expressing a gene encoding a protein of interest; and
recovering the protein of interest.

15. The method of claim 14, wherein the protein of interest is at least one selected from the group consisting of amylases, proteases, xylanases, lipases, laccases, phenol oxidases, oxidases, cutinases, cellulases, hemicellulases, esterases, peroxidases, catalases, glucose oxidases, phytases, pectinases, glucosidases, isomerases, transferases, galactosidases, chitinases, hormones, cytokines, growth factors, receptors, vaccines and antibodies.

16. The method claim 14, wherein the host cell is Kluyveromyces marxianus or Escherichia coli.

17. The method claim 14, wherein the host cell is Kluyveromyces marxianus and produces a protein of interest at a higher level than a precursor host cell of the same type without the expression vector.

18. A method of expressing a gene product in Kluyveromyces, comprising:
transforming a Kluyveromyces cell with the expression vector of claim 8; and
culturing the transformed cell under suitable conditions for expressing the encoded product.

19. The method of claim 18, further comprising recovering the expressed product.
